(19) United States
(12) Patent Application Publication
(10) Pub. No.: US 2006/0155362 A1
(43) Pub. Date: Jul. 13, 2006

(54) Stent with expandable aperture

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/033,855

(22) Filed: Jan. 13, 2005

(51) Int. Cl. A61F 2/06

(52) U.S. Cl. 623/1.15

(57) ABSTRACT

A stent including a stent body constructed of a pattern of filaments, and having a side aperture defined by a contour of a loop constructed of at least one filament connected to the rest of the filaments by filament struts, the stent body being expandable to an expanded configuration wherein the loop and the filament struts expand outwards from the rest of the stent body. In the expanded configuration, the loop may expand in a translatory motion, radially outwards from the rest of the stent body. In a non-expanded configuration, the filament struts may lie generally flat with an outer contour of the stent, and in the expanded configuration, the filament struts may bend outwards of the stent at a non-zero angle with respect to the outer contour of the stent.

### FIELD OF THE INVENTION

The present invention relates generally to stents, and particularly to a stent with a translatory expandable aperture, which may be useful in implantation of bifurcated stents in a body.

### BACKGROUND OF THE INVENTION

There are bifurcated lumens, such as but not limited to, the carotid artery, which may need support with a bifurcated stent in procedures, such as but not limited to, percutaneous transluminal coronary angioplasty (PTCA). A bifurcated lumen (also called bifurcation) is an area of the vasculature where a first vessel is bifurcated into two or more branch vessels. Stenotic lesions may form in or around such bifurcations, that is, in or around one or more of the vessels.

However, delivering and deploying a stent to support a bifurcated lumen is a difficult challenge. Some of the problems include the difficulty of properly orienting the stent with respect to the bifurcation and the difficulty of providing a stent that supports the main trunk and branches of the bifurcation without blocking the passageways or causing turbulence or other flow disruptions.

An example of a prior art bifurcated stent is described in U.S. Pat. No. 6,835,203 to Vardi et al., assigned to Advanced Stent Technologies, Inc. The bifurcating stent includes a generally cylindrical main stent and a generally cylindrical branch stent. The branch stent is attached to a side hole formed in the main stent. This may be accomplished by expanding the main stent, wherein the side hole expands and remains flush with the rest of the main stent. The branch stent is then attached to the side hole by wires that protrude from the end of the branch stent and are secured to the side hole.

Vardi et al. also describes stent structures that have differential radial expansion characteristics. For example, the tubular main stent with the side hole is configured so that a portion of the stent on one side of the side hole will expand at a different yield or threshold force than a portion of the stent on the other side of the side hole. In any event, the branch stent is attached to the side hole as described above.

### SUMMARY OF THE INVENTION

The present invention seeks to provide a stent with a translatory expandable aperture that facilitates implanting bifurcated stents in a body, as is described more in detail hereinbelow.

There is provided in accordance with an embodiment of the present invention a stent including a stent body constructed of a pattern of filaments, and having a side aperture defined by a contour of a loop constructed of at least one filament connected to the rest of the filaments by filament struts, the stent body being expandable to an expanded configuration wherein the loop and the filament struts expand outwards from the rest of the stent body. In the expanded configuration, the loop may expand in a translatory motion, radially outwards from the rest of the stent body. In a non-expanded configuration, the filament struts may lie generally flat with an outer contour of the stent, and in the expanded configuration, the filament struts may bend outwards of the stent at a non-zero angle with respect to the outer contour of the stent.

There may also be provided a branch stent attachable to the loop by means of an attachment device. The attachment device may attach to an inside or outside surface of the loop.

### BRIEF DESCRIPTION OF DRAWINGS

The present invention will be further understood and appreciated from the following detailed description taken in conjunction with the drawing in which:

FIG. 1 is a simplified “rolled-out” view of a stent, constructed and operative in accordance with an embodiment of the invention,

FIG. 2 is a simplified view of the stent of FIG. 1 in tubular form, prior to expansion thereof,

FIG. 3 is a simplified view of the stent of FIG. 1 after expansion thereof, showing a translatory expandable aperture that has expanded outwards; and

FIGS. 4A and 4B are simplified pictorial illustrations of ends of a branch stent which may be attached to the expandable aperture of the stent of FIGS. 1-3, in accordance with different embodiments of the invention.

### DETAILED DESCRIPTION OF EMBODIMENTS

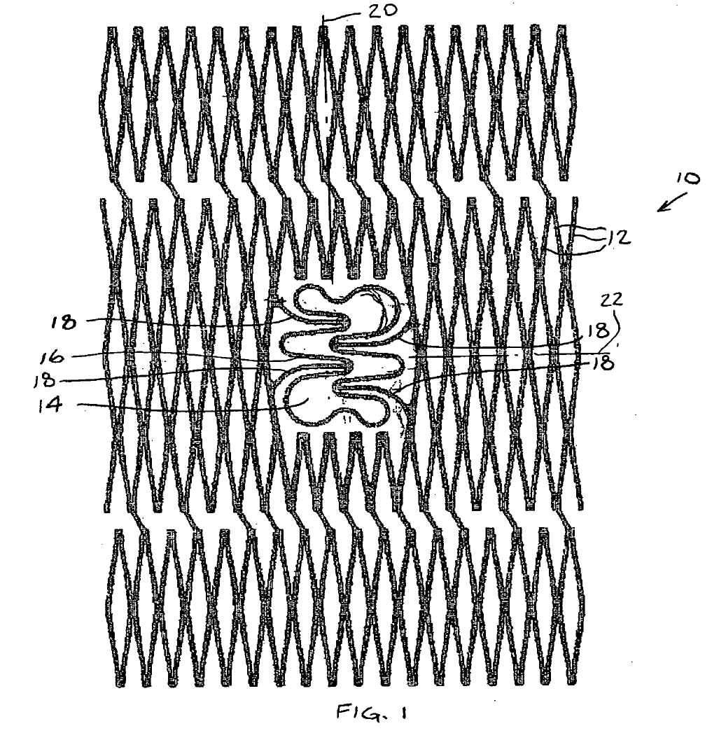

Reference is now made to FIG. 1, which illustrates a stent 10, constructed and operative in accordance with an embodiment of the invention.

Stent 10 may be self-expanding, constructed from a suitable material, such as but not limited to, a shape memory alloy (such as a nickel-titanium alloy, e.g., NITINOL). In the non-limiting illustrated embodiment, stent 10 is a wire mesh stent. However, the invention is not limited to this construction and may also include, among others, a braided stent. Alternatively, without limitation, stent 10 may be balloon-expandable, constructed from a suitable material, such as but not limited to, stainless steel 316L. Stent 10 may be coated, such as a drug-eluting stent that has a polymer coating that emits an anti-restenosis drug.

FIG. 1 shows a non-limiting pattern for forming stent 10. The pattern may be etched or laser cut or made with any other suitable technique. For example, the body of stent 10 may generally include a zigzag, diamond or crossed pattern of filaments 12. The term “filament” throughout the specification and claims encompasses any slender member used to construct stents, such as but not limited to, wires, wire loops, braids, or other slim or thin elements. A side aperture 14 may be defined by the contour of a loop 16, constructed of one or more filaments (which may be one continuous single filament, for example), which is connected to the rest of the stent filaments 12 by filament struts 18. The loop 16 is illustrated as having a serpentine shape, but the invention is not limited to this shape. Loop 16 may be symmetric about a longitudinal axis 20 and/or a transverse axis 22. The filament struts 18 may also be symmetric about longitudinal axis 20 and/or transverse axis 22.

The stent filaments 12 may be expandable axially along longitudinal axis 20 and may also expand radially outwards.

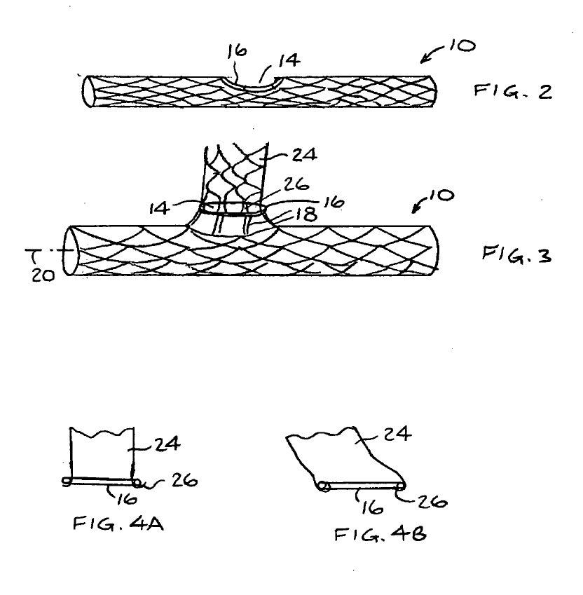

FIG. 2 illustrates stent 10 in its tubular form, prior to expansion thereof. In this configuration, the side aperture 14 may be flush with the rest of the stent 10 (or slightly below or above the outer surface of the stent 10).

Reference is now made to FIG. 3, which illustrates stent 10 after expansion thereof (e.g., self-expansion or balloon expansion). Comparison of FIG. 3 to FIG. 2 shows that side aperture 14 has translated outwards from the outer contour of stent 10. In other words, loop 16, which defines the side aperture 14, has expanded in a translatory motion, radially outwards from the rest of the stent 10. Loop 16 may translate outwards generally parallel to the longitudinal axis 20, or at a non-zero angle with respect to the longitudinal axis 20. The filament struts 18 deform and expand radially outwards. In the non-expanded configuration of FIG. 2, the filament struts 18 lie flat with the outer contour of the stent 10. In the expanded configuration of FIG. 3, the filament struts 18 have been bent outwards of the stent contour and are now perpendicular to the outer contour of the stent 10 (or at some other non-zero angle with respect to the outer contour of the stent 10). Loop 16 and side aperture 14 resemble a “table top” supported by “legs”, which are filament struts 18.

As is well known in angioplasty, stent 10 may be introduced into a body lumen by means of a catheter that passes over a guidewire (not shown), which has been manipulated through vasculature to the site of implanting the stent. Stent 10 may be initially disposed in the catheter in a contracted orientation prior to deployment (in the case of a self-expanding stent) or around expansion balloons of the catheter (in the case of a balloon-expandable stent). Stent 10 may thus be brought to the site of a bifurcation with the side aperture 14 aligned and facing a branch lumen in the body. Upon expansion, side aperture 14 extends into the branch lumen.

Reference is now particularly made to FIGS. 3, 4A and 4B. A branch stent 24 may be attached to loop 16, such as but not limited to, by means of attachment devices 26, e.g., hooks, wire or “petals” at the end of branch stent 24. Branch stent 24 may be introduced into the branch lumen by a guidewire that passes through a side aperture in the catheter (as shown, for example, in U.S. Pat. No. 6,494,905 to Zedler et al.). Branch stent 24 may be introduced into the branch lumen either before or after expanding stent 10. As seen in FIG. 4A, the attachment devices 26 may press and/or hook around the inside of loop 16. Alternatively, as seen in FIG. 4B, the attachment devices 26 may press and/or hook around the outside of loop 16.

As seen in FIG. 4A, the attachment devices 26 may be extend generally straight out of the rest of branch stent 24. Alternatively, as seen in FIG. 4B, the attachment devices 26 may be extend at a non-zero angle (e.g., acute or oblique) from the rest of branch stent 24.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A stent comprising:
a stent body constructed of a pattern of filaments, and having a side aperture defined by a contour of a loop constructed of at least one filament connected to the rest of the filaments by filament struts, the stent body being expandable to an expanded configuration wherein said loop and said filament struts expand outwards from the rest of the stent body.

2. The stent according to claim 1, wherein in the expanded configuration, said loop expands in a translatory motion, radially outwards from the rest of the stent body.

3. The stent according to claim 1, wherein in a non-expanded configuration, said filament struts lie generally flat with an outer contour of the stent, and in the expanded configuration, said filament struts bend outwards of the stent at a non-zero angle with respect to the outer contour of the stent.

4. The stent according to claim 1, wherein said loop has a serpentine shape.

5. The stent according to claim 1, wherein said loop is symmetric about a longitudinal axis of said stent body.

6. The stent according to claim 1, wherein said loop is symmetric about a transverse axis of said stent body.

7. The stent according to claim 1, wherein said stent filaments are expandable axially along a longitudinal axis of said stent body and are also expandable radially outwards.

8. The stent according to claim 1, further comprising a branch stent attachable to said loop by means of an attachment device.

9. The stent according to claim 8, wherein said attachment device attaches to an inside surface of said loop.

10. The stent according to claim 8, wherein said attachment device attaches to an outside surface of said loop.

11. The stent according to claim 8, wherein said attachment device extends generally straight out of the rest of said branch stent.

12. The stent according to claim 8, wherein said attachment device extends at a non-zero angle with respect to the rest of said branch stent.

13. The stent according to claim 8, wherein said branch stent is adapted for introduction into a branch lumen before expanding said stent.

14. The stent according to claim 8, wherein said branch stent is adapted for introduction into a branch lumen after expanding said stent.
